(19) United States
(12) Patent Application Publication
(10) Pub. No.: US 2007/0232550 A1
(43) Pub. Date: Oct. 4, 2007

(54) Use of Hamlet (Human Alpha-Lactalbumin Made Lethal to Tumour Cells) for Treating Viral Infections

(76) Inventor: Catharina Svanborg, Lund (SE)

Correspondence Address: NIXON & VANDERHYE, PC, 901 NORTH GLEBE ROAD, 11TH FLOOR, ARLINGTON, VA, 22203, US

(21) Appl. No.: 11/578,835

(22) PCT Filed: Apr. 20, 2005
(86) PCT No.: PCT/IB05/01255

§ 371 Date: Nov. 22, 2006

(30) Foreign Application Priority Data
Apr. 20, 2004 (GB) 0408752.4

(51) Int. Cl. A61K 38/38

(52) U.S. Cl. 514/21

(57) ABSTRACT

The use of a biologically active complex of α-lactalbumin, selected from HAMLET (human α-lactalbumin made lethal to tumour cells) or a biologically active modification thereof, or a biologically active fragment of either of these, in the preparation of a medicament for use in the treatment of viral infections.

The present invention relates to a method of treatment of viral infections, and to the use of biologically active complexes in the preparation of medicaments for the treatment of viral infections.

HAMLET (human (α-lactalbumin made lethal to tumour cells) (formerly known as MAL) is an active folding variant of alpha-lactalbumin (also represented as α-lactalbumin) that induces apoptosis in transformed cells but spares healthy differentiated cells (M. Svensson, et al., (2000) Proc Natl Acad Sci USA, 97, 4221-6). HAMLET has been shown to bind to the surface of tumour cells, to translocate into the cytoplasm and to accumulate in cell nuclei, where it causes DNA fragmentation (M. Svensson, et al., (2000) Proc Natl Acad Sci USA, 97, 4221-6). Biologically active complexes of this type, obtained from milk and particularly human milk, together with their use as antibacterial agents is described for example in EP-0776214.

To date, work reported with HAMLET has indicated that in-vitro, transformed cells are susceptible to HAMLET, which suggests that there it has an application in cancer therapy (see for example C. Svanborg et al. Advances in Cancer Research. United States, 2003, 88, 1-29). It has also been found to have antibacterial effects, and in particular, inhibits attachment of S. pneumoniae and H. influenzae to human respiratory tract epithelial cells when tested in vitro (WO96/04929). On the contrary, certain viruses, for instance some types of adenovirus, appears to enhance in vitro adherence of certain bacteria (A. Hakansson et al. Infection and Immunity, (1994) vol 62, 7 :2707-2714).

The applicants have found that HAMLET and complexes of this type produce unexpectedly good results when used in the treatment of viral infections.

According to the present invention, there is provided the use of a biologically active complex of α-lactalbumin, selected from HAMLET or a biologically active modification thereof, or a biologically active fragment of either of these, in the preparation of a medicament for use in the treatment of viral infections.

It appears that the effect of HAMLET or biologically active modifications thereof targets specific cell types, and this includes cells, which have been infected with viruses, in addition to the tumour cells, which have been reported previously.

Therefore, these complexes will have application in the treatment of viral infections. If treated in time, HAMLET causes cells that contain a virus such as a retrovirus to die, before the virus has had an opportunity to replicate. As a result, the spread of the virus is contained.

A wide range of viruses may be treated in this way including both human and veterinary viruses. A list of such viruses follows.

1. Family Adenoviridae including the Genus Mastadenovirus, Type species: human adenovirus 2 and the Genus Aviadenovirus, Type species: fowl adenovirus 1 and African swine fever-like viruses

2. Family Arenaviridae including the Genus Arenaviruses, Type species lymphocytic choriomeningitis virus and the Genus Arterivirus, Type species: equine arteritis virus

3. Family Astroviridae which includes the Genus Astrovirus, Type species: human astrovirus 1

4. Family Birnaviridae which includes the Genus Aquabirnavirus, Type species: infectious pancreatic necrosis virus and the Genus Avibirnavirus, Type species: infectious bursal disease virus

5. Family Bunyaviridae which includes the Genus Bunyavirus, Type species: Bunyamwera virus, Genus Hantavirus, Type species: Hantaan virus, Genus Nairovirus, Type species: Nairobi sheep disease virus and Genus Phlebovirus, Type species: sandfly fever Sicilian virus

6. Family Caliciviridae which includes the Genus Calicivirus, Type species: vesicular exanthema of swine virus

7. Family Circoviridae which includes the Genus Circovirus, Type species: chicken anemia virus

8. Family Coronaviridae which includes the Genus Coronavirus, Type species: avian infectious bronchitis virus, the Genus Torovirus, Type species: Berne virus and the Genus Deltavirus, Type species: hepatitis delta virus,

9. Order: Mononegavirales which includes the Family Filoviridae including the Genus: Filovirus, Type species: Marburg virus

10. Family Flaviviridae which includes the Genus Flavivirus, Type species: yellow fever virus, the Genus Pestivirus, Type species: bovine diarrhea virus, and Genus “Hepatitis C-like viruses”, Type species: hepatitis C virus

11. Family Hepadnaviridae which includes the Genus Orthohepadnavirus, Type species: hepatitis B virus and the Genus Avihepadnavirus, Type species: duck hepatitis B virus

12. Family Herpesviridae including its Subfamily: Alphaherpesvirinae for example the Genus Simplexvirus, Type species: human herpesvirus 1, the Genus Varicellovirus, Type species: human herpesvirus 3; and its Subfamily Betaherpesvirinae which includes the Genus Cytomegalovirus, Type species: human herpesvirus 5, the Genus Muromegalovirus, Type species: mouse cytomegalovirus 1, the Genus Roseolovirus, Type species: human herpesvirus 6 and also the Subfamily Gammaherpesvirinae which includes the Genus Lymphocryptovirus, Type species: human herpesvirus 4 and the Genus Rhadinovirus, Type species: ateline herpesvirus 2

13. Family Iridoviridae which includes the Genus Ranavirus, Type species: frog virus 3, the Genus Lymphocystivirus, Type species: flounder virus and the Genus “Goldfish virus-like viruses”, Type species: goldfish virus 1

14. Order: Mononegavirales, including the Familys Filoviridae, Paramyxoviridae, Rhabdoviridae

15. Family Orthomyxoviridae which includes the Genus Influenzavirus A, B, Type species: influenza A virus, the Genus Influenzavirus C, Type species: influenza C virus and the Genus “Thogoto-Like viruses”, Type species: Thogoto virus

16. Family Papovaviridae which includes the Genus Polyomavirus, Type species: murine polyomavirus and the Genus Papillomavirus, Type species: cottontail rabbit papillomavirus (Shope)

17. Order: Mononegavirales including the Family Paramyxoviridae and its subfamily Paramyxovirinae which includes the Genus Paramyxovirus, Type species: human parainfluenza virus 1, the Genus Morbillivirus, Type species: measles virus, the Genus Rubulavirus, Type species: mumps virus; and Subfamily Pneumovirinae including the Genus Pneumovirus, Type species: human respiratory syncytial virus

18. Family Parvoviridae including its Subfamily Parvovirinae which includes the Genus Parvovirus, Type species: mice minute virus, the Genus Erythrovirus, Type species: B19 virus and the Genus Dependovirus, Type species: adeno-associated virus 2

19. Family Picornaviridae which includes the Genus Enterovirus, Type species: poliovirus 1, the Genus Rhinovirus, Type species: human rhinovirus 1A, the Genus Hepatovirus, Type species: hepatitis A virus, the Genus Cardiovirus, Type species: encephalomyocarditis virus and the Genus Aphthovirus, Type species: foot-and-mouth disease virus O

20. Family Poxviridae including its Subfamily Chordopoxvirinae which includes the Genus Orthopoxvirus, Type species: vaccinia virus, the Genus: Parapoxvirus, Type species: orf virus, the Genus Avipoxvirus, Type species: fowlpox virus, the Genus Capripoxvirus, Type species: sheeppox virus, the Genus Leporipoxvirus, Type species: myxoma virus, the Genus Suipoxvirus, Type species: swinepox virus, the Genus Molluscipoxvirus, Type species: Molluscum contagiosum virus and the Genus Yatapoxvirus, Type species: Yaba monkey tumor virus

21. Family Reoviridae which includes the Genus Orthoreovirus, Type species: reovirus 3, the Genus Orbivirus, Type species: bluetongue virus 1, the Genus Rotavirus, Type species: simian rotavirus SA11, the Genus Coltivirus, Type species: Colorado tick fever virus and the Genus Aquareovirus, Type species: golden shiner virus

22. Family Retroviridae which includes the Genus “mammalian type B retroviruses”, Type species: mouse mammary tumor virus, the Genus “mammalian type C retroviruses”, Type species: murine leukemia virus, the Genus “avian type C retroviruses”, Type species: avian leukosis virus, the Genus “type D retroviruses”, Type species: Mason-Pfizer monkey virus, the Genus “blv-htlv retroviruses”, Type species: bovine leukemia virus, the Genus Lentivirus, Type species: human immunodeficiency virus 1 and the Genus Spumavirus, Type species: human spumavirus

23. Order: Mononegavirales including the Family Rhabdoviridae which includes the Genus Vesiculovirus, Type species: vesicular stomatitis Indiana virus, the Genus Lyssavirus, Type species: rabies virus and the Genus Ephemerovirus, Type species: bovine ephemeral fever

24. Satellites including dsDNA Satellites and ssRNA Satellites

25. Family Togaviridae which include the Genus Alphavirus, Type species: Sindbis virus and the Genus Rubivirus, Type species: rubella virus

Specific virus which may be treated using the biological complexes described include viruses of the respiratory tract, gastrointestinal viruses, immunodeficiency viruses such as human immunodeficiency virus (HIV), and viruses of the brain such as viral meningitis or of other internal organs.

The conditions found at mucosal surfaces can be quite unique in terms of properties such as p.H. and the like. However, the complexes of the invention remain active in these conditions. Mucosal surfaces are found inter alia in the nasal passages, in the mouth, throat, oesophagus, lung, stomach, colon, vagina and bladder. Particular mucosal surfaces that may be treated with in accordance with the invention include throat, lung, colon and bladder surfaces. The invention is particularly applicable to the treatment of viruses that affect these areas, such as HSV.

Particular viruses of the respiratory tract which may be treated in accordance with the invention include adenovirus, influenza viruses, respiratory syncytial virus (RSV), parainfluenza, Rhinoviruses, Coronaviruses.

As used herein, the term “HAMLET” refers to a biologically active complex of α-lactalbumin, which is either obtainable by isolation from casein fractions of milk which have been precipitated at pH 4.6, by a combination of anion exchange and gel chromatography as described for example in EP-A-0776214, or by subjecting α-lactalbumin to ion exchange chromatography in the presence of a cofactor from human milk casein, characterized as C18:1 fatty acid as described in WO 99/26979.

The α-lactalbumin may be from various mammalian sources including human, bovine, sheep and goat milk, but is preferably human or bovine, and most preferably human. Recombinant forms of the protein may also be employed.

It has also been found that other reagents and specifically lipids such as oleic acid, are useful in the conversion of human α-lactalbumin to HAMLET. In particular, it has been reported previously that oleic acid (C18:1:9cis) is required for HAMLET production (M. Svensson, et al., (2000) Proc Natl Acad Sci USA, 97, 4221-6). More recently, it has been found that other fatty acids may act as co-factors in a similar way. Optimal cofactors for the conversion of α-lactalbumin to HAMLET are C18:1 fatty acids with a double bond in the cis conformation at position 9 or 11.

α-Lactalbumin is a 14.2 kDa globular protein with four α-helices (residues 1-34, 86-123) and an anti-parallel β-sheet (residues 38-82), linked by four disulphide bonds (61-77; 73-91; 28-111 and 6-120) (K. R. Acharya, et al., (1991) J Mol Biol, 221, 571-81). The native conformation of α-lactalbumin is defined by a high affinity Ca²⁺ binding site, co-ordinated by the side chain carboxylates of Asp82, Asp87 and Asp88, the carbonyl oxygens of Lys79 and Asp84, and two water molecules (K. R. Acharya, et al., (1991) J Mol Biol, 221, 571-81). The protein adopts the so called apo-conformation found in HAMLET when exposed to low pH, or in the presence of chelators, that release the strongly bound Ca²⁺ ion (D. A. Dolgikh, et al., (1981) FEBS Lett, 136, 311-5; K. Kuwajima, (1996) Faseb J, 10, 102-09).

In order to form biologically active complexes, α-lactalbumin generally requires both a conformational or folding change as well as the presence of a lipid cofactor. The conformational change is suitably effected by removing calcium ions from α-lactalbumin. In a preferred embodiment, this is suitably facilitated using a variant of (α-lactalbumin which does not have a functional calcium binding site.

Biologically active complexes which contain such variants are encompassed by the term “modifications” of HAMLET as used herein. However, the applicants have found that, once formed, the presence of a functional calcium binding site, and/or the presence of calcium, does not affect stability or the biological activity of the complex. Biologically active complexes have been found to retain affinity for calcium, without loss of activity. Therefore complex of the invention may further comprise calcium ions.

Thus in particular, the invention uses a biologically active complex comprising alpha-lactalbumin or a variant of alpha-lactalbumin which is in the apo folding state, or a fragment of either of any of these, and a cofactor which stabilises the complex in a biologically active form, provided that any fragment of alpha-lactalbumin or a variant thereof comprises a region corresponding to the region of α-lactalbumin which forms the interface between the alpha and beta domains.

Suitably the cofactor is a cis C18:1:9 or C18:1:11 fatty acid or a different fatty acid with a similar configuration.

In a particular convenient embodiment, the biologically active complex used in the invention comprises (i) a cis C18:1:9 or C18:1:11 fatty acid or a different fatty acid with a similar configuration; and (ii) (α-lactalbumin from which calcium ions have been removed, or a variant of (α-lactalbumin from which calcium ions have been released or which does not have a functional calcium binding site; or a fragment of either of any of these, provided that any fragment comprises a region corresponding to the region of α-lactalbumin which forms the interface between the alpha and beta domains.

As used herein the expression “variant” refers to polypeptides or proteins which are homologous to the basic protein, which is suitably human or bovine α-lactalbumin, but which differ from the base sequence from which they are derived in that one or more amino acids within the sequence are substituted for other amino acids. Amino acid substitutions may be regarded as “conservative” where an amino acid is replaced with a different amino acid with broadly similar properties. Non-conservative substitutions are where amino acids are replaced with amino acids of a different type. Broadly speaking, fewer non-conservative substitutions will be possible without altering the biological activity of the polypeptide. Suitably variants will be at least 60% identical, preferably at least 70%, even more preferably 80% or 85% and, especially preferred are 90%, 95% or 98% or more identity.

When comparing amino acid sequences for the purposes of determining the degree of identity, programs such as BESTFIT and GAP (both from Wisconsin Genetics Computer Group (GCG) software package). BESTFIT, for example, compares two sequences and produces an optimal alignment of the most similar segments. GAP enables sequences to be aligned along their whole length and finds the optimal alignment by inserting spaces in either sequence as appropriate. Suitably, in the context of the present invention when discussing identity of sequences, the comparison is made by alignment of the sequences along their whole length.

The term “fragment thereof” refers to any portion of the given amino acid sequence which will form a complex with the similar activity to complexes including the complete α-lactalbumin amino acid sequence. Fragments may comprise more than one portion from within the full length protein, joined together. Portions will suitably comprise at least 5 and preferably at least 10 consecutive amino acids from the basic sequence. Suitable fragments will be deletion mutants suitably comprise at least 20 amino acids, and more preferably at least 100 amino acids in length. They include small regions from the protein or combinations of these.

The region which forms the interface between the alpha and beta domains is, in human α-lactalbumin, defined by amino acids 34-38 and 82-86 in the structure. Thus suitable fragments will include these regions, and preferably the entire region from amino acid 34-86 of the native protein.

In a particularly preferred embodiment, the biologically active complex comprises a variant of α-lactalbumin in which the calcium binding site has been modified so that the affinity for calcium is reduced, or it is no longer functional.

It has been found that in bovine α-lactalbumin, the calcium binding site is coordinated by the residues K79, D82, D84, D87 and D88. Thus modification of this site or its equivalent in non-bovine α-lactalbumin, for example by removing one of more of the acidic residues, can reduce the affinity of the site for calcium, or eliminate the function completely and mutants of this type are a preferred aspect of the invention.

The Ca²⁺-binding site of bovine α-lactalbumin consists of a 3₁₀ helix and an α-helix with a short turn region separating the two helices (Acharya K. R., et al., (1991) J Mol Biol 221, 571-581). It is flanked by two disulfide bridges making this part of the molecule fairly inflexible. Five of the seven oxygen groups that co-ordinate the Ca²⁺are contributed by the side chain carboxylates of Asp82, 87 and 88 or carbonyl oxygen's of Lys79 and Asp84. Two water molecules supply the remaining two oxygen's (Acharya K. R., et al., (1991) J Mol Biol 221, 571-581). Site directed mutagenesis of the aspartic acid at position 87 to alanine (D87A) has previously been shown to inactivate the strong calcium-binding site (Anderson P. J., et al., (1997) Biochemistry 36, 11648-11654) and the mutant proteins adopted the apo- conformation.

Therefore in a particular embodiment, the aspartic acid residue at amino acid position 87 within the bovine α-lactalbumin protein sequence is mutated to a non-acidic residue, and in particular a non-polar or uncharged polar side chain.

Non-polar side chains include alanine, glycine, valine, leucine, isoleucine, proline, phenylalanine, methionine, tryptophan or cysteine. A particularly preferred examples is alanine. Uncharged polar side chains include asparagine, glutamine, serine, threonine or tyrosine.

In order to minimize the structural distortion in the mutant protein, D87 has also been replaced by an asparagine (N) (Permyakov S. E., et al., (2001) Proteins Eng 14, 785-789), which lacks the non-compensated negative charge of a carboxylate group, but has the same side chain volume and geometry. The mutant protein (D87N) was shown to bind calcium with low affinity (K−_{ca}2×10⁵ M⁻¹) (Permyakov S. E., et al., (2001) Proteins Eng 14, 785-789). Such a mutant forms an element of the biologically active complex in a further preferred embodiment of the invention.

Thus particularly preferred variants for use in the complexes of the invention are D87A and D87N variants of α-lactalbumin, or fragments which include this mutation.

This region of the molecule differs between the bovine and the human proteins, in that one of the three basic amino acids (R70) is changed to S70 in bovine α-lactalbumin thus eliminating one co-ordinating side chain. It may be preferable therefore, that where the bovine α-lactalbumin is used in the complex of the invention, an S70R mutant is used.

The Ca²⁺ binding site is 100% conserved in α-lactalbumin from different species (Acharya K. R., et al., (1991) J Mol Biol 221, 571-581), illustrating the importance of this function for the protein. It is co-ordinated by five different amino acids and two water molecules. The side chain carboxylate of D87 together with D88 initially dock the calcium ion into the cation-binding region, and form internal hydrogen bonds that stabilise the structure (Anderson P. J., et al., (1997) Biochemistry 36, 11648-11654). A loss of either D87 or D88 has been shown to impair Ca2+ binding, and to render the molecule stable in the partially unfolded state (Anderson P. J., et al., (1997) Biochemistry 36, 11648-11654).

Further, mutant proteins with two different point mutations in the calcium-binding site of bovine α-lactalbumin may be used. For example, substitution of the aspartic acid at position 87 by an alanine (D87A) has been found to totally abolish calcium binding and disrupt the tertiary structure of the protein. Substitution of the aspartic acid by asparagine, the protein (D87N) still bound calcium but with lower affinity and showed a loss of tertiary structure, although not as pronounced as for the D87A mutant (Permyakov S. E., et al., (2001) Proteins Eng 14, 785-789). The mutant protein showed a minimal change in packing volume as both amino acids have the same average volume of 125 Å3, and the carboxylate side chain of asparagines allow the protein to co-ordinate calcium, but less efficiently (Permyakov S. E., et al., (2001) Proteins Eng 14, 785-789). Both mutant proteins were stable in the apo-conformation at physiologic temperatures but despite this conformational change they were biologically inactive. The results demonstrate that a conformational change to the apo-conformation alone is not sufficient to induce biological activity.

The structure of α-lactalbumin is known in the art, and the precise amino acid numbering of the residues referred to herein can be identified by reference to the structures shown for example in Anderson et al. supra. and Permyakov et al supra. The medicaments produced in accordance with the invention are suitably pharmaceutical compositions in a form suitable for topical administration to the particular area which is infected by the virus.

For instance, for infections of the respiratory tract, the composition may be suitable for administration by inhalation or insufflation. Oral compositions may be used to treat viral infections of the gastrointestinal tract, in particular of stomach infections. Treatment of viral infections of other mucosal surfaces, such as the vagina or colon, may be treated with topical formulations such as suppositories.

Topical solutions or creams suitably contain an emulsifying agent for the protein complex together with a diluent or cream base may be more suitable for application to other viral infections at mucosal surfaces. Such formulations can be applied directly to the surface.

In addition, such topical compositions may be applied to treat viral infections of the skin.

In another example, the composition may be in a form which is suitable for instillation into the bladder, where a bladder virus is the being treated.

Other viral infections that affect internal organs may be treated by infusion into the affected area. Such areas may include the brain, liver, kidney, prostate and ovaries. Similarly immunodeficiency viruses may be treated by infusing the complex into the organs responsible for the immune system, such as the lymph glands and bone marrow.

The complex is suitably infused using convection enhanced delivery (CED).

The compositions may include the commonly known carriers, fillers and/or expedients, which are pharmaceutically acceptable.

For instance, compositions which are instilled or infused into the bladder or other internal organs will comprise a solution of the active agent in sterile water or saline.

The active reagent in accordance with the invention will attack virus infected cells, causing them to die, probably by apoptosis, but will spare healthy cells. As a result, the spread of the virus will be curtailed.

The daily dose of the active compound varies and is dependant on the patient, the nature of the virus being treated etc. in accordance with normal clinical practice. As a general rule from 200 mg to lg/dose of the biologically active complex is used for administration per day, over a period of at least 3 and preferably at least 5 days.

In a further aspect of the invention, there is provided a method for treating viral infections which comprises administering to a patient in need thereof, a biologically active complex of α-lactalbumin, selected from HAMLET or a biologically active modification thereof, or a biologically active fragment of either of these.

In particular, the complex will be administered directly to virus infected cells. For instance, for treating viral infections of mucosal surfaces, the complex will be administered to said surface in a therapeutically effective amount.

Preferred examples of the biologically active complex are illustrated above. Preferably the biologically active complex is administered in the form of a topical composition, also as described above.

1. A method of treating a viral infection comprising administering biologically active complex of α-lactalbumin, selected from HAMLET or a biologically active modification thereof, or a biologically active fragment of either of these, to a person in need of said treatment.

2. The method of use wherein the viral infection is an infection of the respiratory tract, gastrointestinal viruses, immunodeficiency viruses, and viruses of the brain or of other internal organs.

3. The method of claim 1 wherein the biologically active complex comprising alpha-lactalbumin or a variant of alpha-lactalbumin which is in the apo folding state, or a fragment of either of any of these, and a cofactor which stabilises the complex in a biologically active form, provided that any fragment of alpha-lactalbumin or a variant thereof comprises a region corresponding to the region of α-lactalbumin which forms the interface between the alpha and beta domains.

4. The method of claim 3 wherein the cofactor is a cis C18:1:9 or C18:1:11 fatty acid or a different fatty acid with a similar configuration.

5. The method of claim 1 wherein the biologically active complex comprises HAMLET, which is obtainable either by isolation from casein fractions of milk which have been precipitated at pH 4.6, by a combination of anion exchange and gel chromatography, or by subjecting α-lactalbumin to ion exchange chromatography in the presence of a cofactor from human milk casein, characterized as C18:1 fatty acid.

6. The method of claim 1 wherein the biologically active complex of α-lactalbumin comprises
(i) a cis C18:1:9 or C18:1:11 fatty acid ora different fatty acid with a similar configuration; and
(ii) α-lactalbumin from which calcium ions have been removed, or a variant of α-lactalbumin from which calcium ions have been removed or which does not have a functional calcium binding site; or a fragment of either of any of these, provided that any fragment comprises a region corresponding to the region of α-lactalbumin which forms the interface between the alpha and beta domains.

7. The method of claim 6 wherein the biologically active complex includes a variant of α-lactalbumin in which the calcium binding site has been modified so that the affinity for calcium is reduced, or it is no longer functional.

8. The method of claim 7 wherein the variant has a mutation at one of the amino acids equivalent to K79, D82, D84, D87 and D88 of bovine α-lactalbumin.

9. The method of claim 8 wherein the modification is at D87 which includes a variant of α-lactalbumin having a D87A or D87N variants.

10. The method of claim 1 wherein the biologically active complex comprises a fragment of α-lactalbumin or a variant thereof, and where the fragment includes the entire region from amino acid 34-86 of the native protein.

11. The method of claim 1 wherein the (α-lactalbumin is human or bovine α-lactalbumin or a variant of either of these.

12. The method of wherein the α-lactalbumin is human α-lactalbumin.

13. The method of claim 11 wherein the α-lactalbumin is mutant bovine α-lactalbumin which includes an S70R mutation.

14. A method for treating viral infections which comprises administering to a patient in need thereof, a biologically active complex of α-lactalbumin, selected from HAMLET or a biologically active modification thereof, or a biologically active fragment of either of these.

15. A method according to claim 14 for treating a viral infection at a mucosal surface which comprises administering to said surface in a patient in need thereof, a biologically active complex of α-lactalbumin, selected from HAMLET or a biologically active modification thereof, or a biologically active fragment of either of these.

16. A method of producing a medicament comprising formulating a biologically active complex of α-lactalbumin, selected from HAMLET or a biologically active modification thereof, or a biologically active fragment of either of these.
